(19) United States
(12) Patent Application Publication
(10) Pub. No.: US 2007/0088343 A1
(43) Pub. Date: Apr. 19, 2007

(54) Flexible surgical shaft having grasping sections

(76) Inventors: John McIntyre, Foothill Ranch, CA (US); Tom Gass, Phoenix, AZ (US)

Correspondence Address: STETINA BRUNDA GARRED & BRUCKER, 75 ENTERPRISE, SUITE 250, ALISO VIEJO, CA, 92656, US

(21) Appl. No.: 11/519,689

(22) Filed: Sep. 11, 2006

Related U.S. Application Data
(60) Provisional application No. 60/718,912, filed on Sep. 19, 2005.

(51) Int. Cl. A61B 18/18

(52) U.S. Cl. 606/13

(57) ABSTRACT

The present invention contemplates an elongated surgical guide shaft for placement within a body and surgical devices containing such a guide shaft. The shaft includes both a flexible portion and a gripping portion. The gripping portion is adapted to be handled or gripped by surgical tools and is resistant to crushing. The present invention also contemplates a method for performing a minimally invasive surgery. The method includes inserting a surgical device including such a guide shaft into a patient, grasping the gripping section of the guide shaft with a surgical tool in order to aid proper placement of the guide shaft, and using the surgical device to treat the patient.

### CROSS-REFERENCE TO RELATED APPLICATIONS

This application claims the benefit of U.S. Provisional Application No. 60/718,912, filed on Sep. 19, 2005, the teachings of which are expressly incorporated by reference.

### STATEMENT RE: FEDERALLY SPONSORED RESEARCH/DEVELOPMENT

Not Applicable

### BACKGROUND

1. Field of the Invention

The present invention relates to the field of surgery and more particularly to an elongated flexible surgical apparatus having graspable sections for use in minimally invasive surgical procedures.

2. Background of the Invention

Medical science has, developed a wide variety of surgical devices for accessing different areas of the body and for performing various surgical procedures. Many of these surgical devices and procedures require a relatively large opening within the patient's body just to gain access into the body cavity and to the desired area of treatment. This is particularly true with surgical devices having broad and elongated shapes and configurations and when used in procedures requiring movement of the device within the patient or requiring multiple treatment locations. Improvements are continuously being sought to reduce the negative effects of any surgery, including the effects and injuries incurred when delivering the surgical device from the entry location to the treatment areas. This concern has lead to the desire to reduce such effect and further to utilize minimally invasive procedures wherever possible.

The advent of minimally invasive surgical procedures has dramatically altered numerous surgical procedures and even allowed procedures never before possible. In minimally invasive surgery, trauma to the body is reduced, in part, by reducing the surgical opening and passageway required into the body cavity as well as by using specifically designed surgical devices. Thus, there is a great desire to modify existing open surgical devices for application with minimally invasive procedures and ports.

Transmyocardial revascularization (“TMR”) is a procedure wherein energy is delivered to a region of the heart in order to create small channels across the wall of the heart. The procedure is typically performed in patients suffering from severe angina. TMR is performed in a surgical setting in which access to the left ventricle is typically gained through an open surgical sternotomy or a thoracotomy. With the advent of minimally invasive thoracoscopic surgical procedures in recent years, it is desirable to deliver TMR therapy minimally invasively via ports. Similar procedures are contemplated for revascularizing other body tissues. Present TMR surgical devices, however, require unique configurations that generally preclude the use of such minimally invasive ports. Moreover, present devices are subject to damage when delivered through a port due to handling by surgical instruments.

The advent of smaller and smaller surgical devices, however, has lead to numerous challenges, including properly locating the surgical device at the desired location within the body and delivering a surgical treatment device to difficult to reach locations within the body. For example, during minimally invasive surgery, including TMR and related applications, a surgeon must insert an elongated shaft of the desired surgical device into the minimally invasive port and then move it to and position it at the desired location within the body cavity. Typically, the surgeon uses various thoroscopic and related tools to assist with such placement and positioning. These tools, however, can be very difficult to use in conjunction with the flexible elongated shafts and can distort, damage, and even break such devices. In addition, the flexible materials can be difficult to locate and grasp using such surgical tools.

### BRIEF SUMMARY

The present invention comprises a surgical device for treating living tissue within a body. The surgical device of the present invention includes a handle portion for gripping and manipulation by the surgeon or other user. An elongated flexible guide shaft is connected to the handle portion at its proximal end and extends outwardly into a distal portion. The guide shaft includes at least one section that is adapted for handling by a surgical tool and to be resistant to crushing and damage when handled by surgical grasping and handling tools.

In another embodiment of the invention, the distal end of the guide shaft is adapted for making a contact surface with a desired region of the heart and includes a treatment assembly that is adapted for the desired treatment. The handle assembly includes an advancement mechanism to move the treatment assembly outwardly relative to the guide shaft. The advancement mechanism allows the treatment assembly to be translated along the distal portion of the guide shaft so as to translate outwardly from it and also to be retracted.

The present invention further comprises a minimally invasive procedure for treating a patient. The method comprises providing a surgical device having a flexible elongated tubular guide tube with a proximal portion in connection with a handle assembly and a distal portion extending distally from the handle into a treatment tip and wherein the distal portion includes at least one gripping section adapted for being grasped by a surgical tool without effecting the integrity of the guide tube. The distal end of the guide tube is introduced into a first port placed in the patient. The guide tube is then moved by using a surgical tool to grasp a gripping section of the guide tube within the patient and positioning the treatment tip to a desired location within the patient. The surgical device is then used so as to treat the patient.

The present invention further comprises a system and method of surgical myocardial revascularization of the myocardium of the heart of a patient. In this procedure, a surgical opening and preferably a minimally invasive port is created within the patient for access to the treatment area. An elongated flexible surgical apparatus configured into an insertion configuration is inserted into the surgical opening and directed into the chest cavity. The surgical apparatus preferably includes a lasing mechanism in connection with a surgical lasing tip located at the distal end of an elongated flexible guide shaft with the guide shaft including at least one section that is strengthened and adapted for handling by surgical tools.

The guide tube, including the lasing tip is then guided within the patient by using surgical tools grasping the gripping section of the guide shaft and directing the lasing tip into a desired area within the chest cavity. Preferably, the surgical tools area also introduced within the body through ports. Once positioned, the heart is next irradiated with laser energy emitted from the lasing apparatus with sufficient energy and for a sufficient time to cause a channel to be formed from the exterior surface of the epicardium through the myocardium and the epicardium.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of an embodiment of the surgical device of the present invention; and

FIG. 2 is a side view of a preferred embodiment of a section of the guide shaft of the present invention.

### DETAILED DESCRIPTION

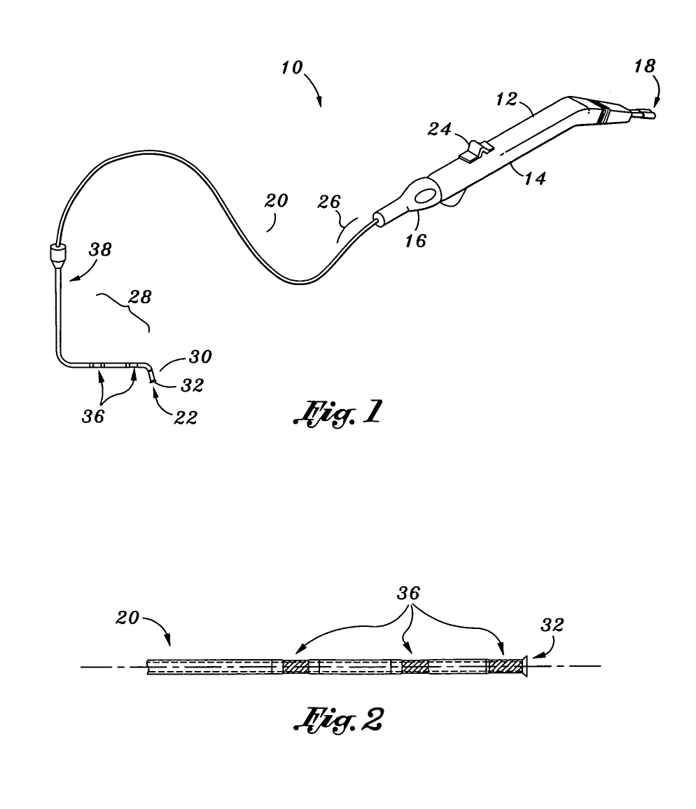

While a variety of embodiments of the present invention are disclosed herein, one exemplary and the presently preferred embodiment of the surgical device is illustrated generally as reference number 10 in FIG. 1. This embodiment of the surgical device 10 is particularly suitable for procedures for treating the heart and other internal tissues, including Transmyocardial revascularization (“TMR”), biopsies, and related procedures. The device 10 is adapted for hand use and manipulation and may be held in several positions using one or both hands.

A preferred method of using the device 10 of the present embodiment involves using a treatment assembly associated with the device to perforate the desired tissue, for example, the epicardium of the heart to create revascularization pathways. In TMR procedures, these pathways are typically revascularization channels which extend into myocardium and may or may not communicate with the ventricle.

Referring now to FIG. 1, the preferred mechanical surgical device 10 includes a hand piece assembly 12 which includes a housing and a mechanism for manipulating at least a portion of a treatment assembly. Preferably the housing is molded or machined from a lightweight plastic material and defining a contoured surface 14 defining one or more finger grip indentations. Preferably, the contoured surface 14 provides tactile feedback regarding the position of the hand on the device so the physician need not look away from the medical procedure or other task at hand. The contoured surface 14 further assists the user to securely hold the hand piece without slippage in at least two different positions during either left or right handed operation of the device 10. A neck portion 16 extends from the distal end of the hand piece 12. In one embodiment, the neck portion is a separate component from the hand piece 12 to allow for a rotary connection. The neck portion 16 may also be constructed from a molded or machined plastic, or similar to the hand piece 12, may be constructed from other materials such as metal or composite materials.

The surgical device 10 generally includes a continuous passageway along its generally longitudinal axis for supporting a treatment assembly 18. In the preferred embodiment for a TMR procedure, the treatment assembly 18 includes an optical fiber bundle whose proximal end is connected to a laser energy source (not shown) and distal end terminates into a treatment end or tip 22. The treatment assembly 18 is moveable along the device's passageway, preferably using a mechanical finger slide 24 configured as part of the handle portion 12, but may also be moveable using most any form of mechanical or electromechanical slide mechanism or may even be moved using an automated mechanism. Through movement of the treatment assembly 18, the treatment end 22 may be extended and retracted. Alternatively, part of the treatment assembly may be fixed relative to the handle 12 with only the distal portion or even the treatment end 22 being adjustable. The present invention further contemplates a handle 12 without a passageway and a treatment assembly 18 that is generally fixed relative to the handle 12.

A flexible elongated guide shaft 20 extends outwardly from a proximal end 26 in connection with the neck portion 16 of the handle 12 into a distal portion 28 and ultimately a distal end 30. The guide shaft 20 is an elongated tubular piece that is constructed of a flexible tubular material having at least one strengthened or gripping section 36 along the distal portion 28. In the preferred embodiment for TMR, the guide shaft 20 is about 60 cm in length, but this length could be varied for any particular application.

The guide shaft 20 includes a lumen having an opening or diameter sufficient to allow passage of the desired treatment apparatus 18. Alternatively, the lumen may be adapted for the treatment apparatus 18 along with other devices, treatment tips or the like. Alternatively, the guide shaft 20 may include multiple lumens adapted for surgical procedures in which multiple treatment devices or passageways are required. For example, the guide shaft 20 may include two lumens, one for the treatment apparatus 18 and one for supplying an alternative treatment to the desired region. Desired treatments may include medication, pain treatment, wound healers, stem cells and the like.

Preferably, the guide shaft 20 is made from a flexible stainless steel coil lined with a flexible, low friction, and surgically suitable polymer such as Teflon or PTFE. The lined coiled material is adapted to allow for passage of the treatment assembly 18 while retaining flexibility and precluding flaking or chipping of the inner lining or treatment assembly 18 passing through it. The stainless steel coil is then reinforced with an outer polymer shaft having varying rigidity. The distal portion of the elongated shaft is then preferably coated with a soft flexible polymer for enhanced gripping characteristics and to prevent flaking and chipping of the hardened sections.

Preferably, the polymer shaft is made from a medical grade polyether block co-polymide polymer (“Pebax”) material providing flexibility and some strength with the external polymer layer providing a smooth surface to facilitate placement within the body and maintaining surgical cleanliness. To provide for strengthened gripping sections 36 along the guide shaft 20, the reinforcing shaft is preferably constructed from a variable durometer Pebax material.

The harder sections 36 are adapted to be crush resistant and are about 0.25 to 2.0 centimeters (“cm”) in length. In a preferred embodiment, the gripping sections 36 of the elongated shaft 20 are between 0.5 and 1.0 cm in length and are separated by more flexible sections of the guide shaft that extend approximately 0.5 to 5.0 cm in length. The crush resistant zones or gripping sections 36 can be colored, color coated, or otherwise marked to enable a surgeon to easily identify those sections 36 that are designed to be grasped with the external manipulation tools. The more flexible regions of the shaft 20 are preferably of lower durometer material than the gripping sections 36.

The hardened materials of the gripping sections 36 are adapted to prevent flaking, chipping, or gouging while being handled by the external tools. Preferably, this is accomplished by coating the external portion of the distal end 30 or the entire guide shaft 20 with a soft coating. Preferably, the coating is a soft clear Pebax. Other suitable materials may include polyurethane, Teflon, silicone, and the like. Alternatively, only the crush resistant or gripping sections 36 are coated.

In the preferred embodiment shown, a stabilizing tip or cup 32 is connected to the distal end 30 of the guide shaft 20. The cup 32 is generally cup or disc shaped and is designed to contact tissue and maintain contact of the distal end 30 on the region of tissue being treated. The stabilizing cup advantageously aligns the treatment tip in the desired orientation and in TMR procedures is adapted to ensure the treatment tip 22 enters the heart tissue at a perpendicular orientation. The stabilizing tip 32 may be constructed from generally yieldable materials such as silicone, soft elastic, rubber, or foam and may also be metallic or plastic. The stabilizing tip 32 may be part of or permanently attached to the distal end 30 of the shaft 20 or may be detachable with conventional snap-mount or screw-mount mechanisms. Different detachable stabilizing tips 32, such as suction and drug delivery tips, may be provided to accommodate different treatment procedures as well as for use through differing access ports.

The distal end or tissue contacting surface of the tip 32 may be textured to provide a gripping surface and suction may be provided at the proximal end of the hand piece to extend through the shaft 20 to further secure the stabilizing tip 26 to the tissue being treated, including the heart. The stabilizing tip 32 preferably includes a bore aligned and in connection with the lumen opening at the distal end 30 of the guide shaft 20. In this way, the treatment assembly 18 may freely pass through the guide shaft 20 and the stabilizing tip 32.

In the preferred embodiment configured for TMR, the proximal end of the cup 32 is rigidly bonded to the distal end 30 of the guide shaft 20 and is configured with a proximal end having a diameter similar to that of the distal end 30 of the guide shaft 20. The cup 32 tapers outwardly from the guide shaft 20 into a contact surface outer diameter of about 5 mm. The vacuum cup 32 is preferably made from a medical grade polyether, such as Pebax, or other medical grade nylon-type material that is sufficiently pliable to form a contact surface with the tissue being treated and is bonded to the distal end 30 of the guide shaft 20. Alternatively, the cup 32 and the outer reinforcing shaft of the guide shaft 20 may be formed from a single piece of flexible plastic material such as Pebax.

The present invention advantageously decreases the maximum outer diameter of the surgical device that must penetrate the patient, as well as provides increased handling capabilities. The smaller diameter facilitates use of the device 10 with smaller and minimally invasive ports.

The device 10 may also be used in conjunction with other surgical components or devices to accomplish the desired surgical procedure. For example, an introducer 38 may be used along the guide shaft 20 to allow for insufflation of the patient cavity during the desired procedure. Various ports may also be used to facilitate introduction and handling of the device 10.

Referring now to FIG. 2, a preferred embodiment of the distal portion 26 of the guide shaft 20 is shown. This embodiment shows three gripping sections 36 between the more flexible sections of the guide shaft 20. It should be understood that any number of gripping sections is contemplated herein, including gripping sections dispersed along the entire length of the guide shaft 20. The invention contemplates sufficient and identifiable gripping regions or sections to allow a surgeon to properly insert the guide shaft 20 and place the treatment end 22. The invention also contemplates the use of various materials to provide the necessary flexibility and hardness for the gripping regions 36 for an applicable procedure.

During a TMR procedure using the surgical device 10 of the present invention, energy is applied to myocardial tissue of the heart by means of the treatment assembly 18 supported within the passageway extending through the hand piece 12 and further extending through the elongated flexible guide shaft 20 and out from the locating cup 32. In the currently preferred procedure, a laser provides the energy that is directed through the treatment assembly 18 which includes a means of carrying the laser energy to the treatment tip 22. In the preferred embodiment, the treatment assembly 18 includes a fiber optic cable to deliver laser energy to the treatment site.

To facilitate a minimally invasive surgical procedure using the present surgical device 10, including the use of ports placed in the patient, the elongated guide shaft or tube 20 is inserted into a port. Because the guide shaft 20 is flexible and without rigidity and is not steerable, the procedure requires that other instruments be used to fully insert the guide shaft 20 into the patient and to position the distal end 30 to the desired location within the patient. Surgical tools such as forceps, graspers, and similar grasping and handling tools are used to grasp a gripping section 36 located along the distal portion 28 of the guide shaft 20. Using the surgical tools, the user can then manipulate the surgical device 10, and particularly the guide shaft 20, within the patient. The surgical tool or tools can be inserted into the patient through surgical incisions or preferably, ports. Similarly, a camera can be inserted into the patient to assist in locating and directing the guide shaft 20 and positioning the treatment end 22 within the patient. Once the guide shaft 20 and treatment end 22 are appropriately positioned, treatment can begin.

In a presently preferred TMR procedure, the distal portion of the guide shaft 20 is inserted into the chest cavity of a patient. This insertion is preferably made through a port entry within the patient such as a thoracoport of about 5 mm in diameter. A thorascopic surgical tool or tools may then be used to grasp a strengthened section 36 of the guide shaft 20 and manipulate the distal end 30 and position the treatment end 22 to the desired location against the heart wall. The flexible nature of the guide shaft 20 advantageously allows for all portions of the heart to be accessed and treated.

The surgical tools may be inserted into ports made at other locations in the patient. In addition, a camera or cameras may be used to assist the surgeon in finding and grasping the guide shaft 20 and in positioning the treatment assembly 22. Once positioned adjacent the epicardium, the surgeon adjusts the treatment assembly 22 such that the treatment tip 30 is positioned generally perpendicular to the epicardium. Once positioned, the treatment assembly may be activated so as to treat the patient.

Further details of the present invention, including various methods of using the present invention may be found with reference to the Detailed Description of Embodiment section of U.S. Pat. No. 5,713,894 issued on Feb. 3, 1998 to Murphy-Chutorian and Harman and to the Detailed Description of the Preferred Embodiment section of U.S. Pat. No. 5,976,164 issued on Nov. 2, 1999 to Bencini, et al., both of which are incorporated in their entirety herein by reference.

The foregoing describes the features and benefits of the present invention in various embodiments. Those of skill in the art will appreciate that the present invention is capable of various other implementations and embodiments that operate in accordance with the foregoing principles and teachings. For example, many of the components may be made from various materials and may be interconnected in various ways. Moreover, the arrangement of an elongated guide shaft for supporting a treatment means may be accomplished by using different tubular shapes or configurations and the use of strengthened gripping regions or sections may be arranged in any number of methods and configurations. This is particularly contemplated as each surgical procedure may require a different treatment means and also differing surgical procedures and thus differing gripping section configurations and locations along the guide shaft. The handle may be made of materials other than plastic and may be configured differently to provide alternative designs. Accordingly, this detailed description is not intended to limit the scope of the present invention, which is to be understood by reference to the claims below.

What is claimed is:

1. A device for performing a surgical procedure within a body, comprising:
a control portion; and
a flexible elongated guide tube having a proximal end in connection with the control portion and extending into a distal end wherein the guide tube has a strengthened section adapted for handling by a surgical instrument.

2. The device of claim 1 further comprising a treatment element extending through at least a portion of said guide tube.

3. The device of claim 2 wherein the treatment element comprises an optical fiber for delivering laser energy to a treatment tip.

4. The device of claim 1 wherein the guide tube comprises an elongated coiled spring having a flexible coating.

5. The device of claim 4 wherein the coating is a soft polymer material.

6. The device of claim 1 wherein the strengthened section comprises a hard polymer.

7. The device of claim 6 wherein the hard polymer comprises a Pebax material.

8. The device of claim 1 wherein the strengthened section comprises a strengthened sleeve over the guide tube.

9. The device of claim 1 wherein the strengthened section is colored so as to distinguish as a strengthened section.

10. The device of claim 1 wherein the strengthened section comprises a longitudinal length of between 0.25 cm and 2.0 cm.

11. The device of claim 1 wherein the guide tube comprises an elongated stainless steel coil and the strengthened section comprises a hard polymer coating over said steel coil.

12. The device of claim 1 wherein the control portion is a handle assembly adapted for use by a surgeon.

13. The device of claim 1 wherein the guide tube comprises multiple strengthened sections along its longitudinal axis.

14. The device of claim 3 adapted for delivering pulses of laser energy to the patient's heart.

15. A minimally invasive procedure for treating a patient, comprising the steps of:
a) providing a surgical device having a flexible elongated tubular guide tube with a proximal portion in connection with a handle and a distal portion extending distally from the handle into a treatment tip and wherein the distal portion includes at least one gripping section adapted for being grasped by a surgical tool without effecting the integrity of the guide tube;
b) introducing the distal end of the guide tube into a first port placed in the patient;
c) using a surgical tool to grasp a gripping section of the guide tube within the patient;
d) positioning the treatment tip to a desired location within the patient; and
e) using the surgical device so as to treat the patient.

16. The method of claim 15 wherein the surgical device further comprises an optical transmission fiber translatable within the guide tube for delivering a laser treatment at the treatment tip.

17. The method of claim 16 further comprising the step of providing the laser treatment to the desired location within the patient.

18. An elongated surgical guide tube extending along a longitudinal axis, said guide tube for delivering a treatment device to a desired location within a body comprising:
a flexible portion extending along the longitudinal axis; and
a gripping portion in connection with the flexible portion and adapted for handling by a surgical tool, said gripping portion extending along the longitudinal axis;
wherein the gripping portion comprises a material resistant to crushing.

19. The guide tube of claim 18 wherein the gripping portion further comprises an identifier such that a surgeon can distinguish the gripping portion from other portions of the guide tube.

20. The guide tube of claim 19 wherein the identifier comprises a coloring.

21. The guide tube of claim 18 comprising a series of flexible portions extending along the longitudinal axis separated by at least one gripping portion.

22. An elongated surgical guide shaft for placement within a body comprising:
a flexible portion extending along the longitudinal axis; and
a gripping portion in connection with the flexible portion and adapted for handling by a surgical tool, said gripping portion extending along the longitudinal axis;
wherein the gripping portion comprises a material resistant to crushing.

23. The guide shaft of claim 22 wherein the gripping portion further comprises a coating.

24. The guide shaft of claim 23 wherein the coating is on the external surface of the gripping portion to reduce flaking and damage to said gripping portion.

25. The guide shaft of claim 23 wherein the coating is a Pebax material.

26. The guide shaft of claim 23 wherein the coating is translucent.
